(19) United States
(12) Patent Application Publication
(10) Pub. No.: US 2023/0346726 A1
(43) Pub. Date: Nov. 2, 2023

(54) IBUPROFEN CONTAINING SOFT GELATINE CAPSULE

(71) Applicant: RECKITT BENCKISER HEALTH LIMITED, Berkshire (GB)

(72) Inventors: Georgia Kerry BROWN, Humberside (GB); Matthew Edward Anthony MCGIRR, Humberside (GB); Lucy Amber PHILLIPS, Humberside (GB)

(73) Assignee: RECKITT BENCKISER HEALTH LIMITED, Berkshire (GB)

(21) Appl. No.: 17/999,073

(22) PCT Filed: May 21, 2021
(86) PCT No.: PCT/GB2021/051242

§ 371 Date: Nov. 17, 2022

(30) Foreign Application Priority Data
May 21, 2020 (GB) 2007620.4

(51) Int. Cl. A61K 31/192; A61K 9/48

(52) U.S. Cl. CPC A61K 31/192; A61K 9/4825; A61K 9/4858; A61K 9/4866; A61K 9/485

(57) ABSTRACT

The present invention relates to a composition for encapsulation in a soft gelatin shell which comprises ibuprofen, one or more polyvinyl pyrrolidones and one or more polyethylene glycols wherein the weight ratio of the ibuprofen to the one or more polyvinylpyrrolidones is from 2:1-15:1, the weight ratio of the ibuprofen to the one or more polyethylene glycols is from 1:1-5:1 and the weight ratio of the one or more polyethylene glycols to the one or more polyvinylpyrrolidones is 2:1-10:1.

The present invention is directed to a composition that comprises ibuprofen. In particular, the present invention is directed to a composition that comprises ibuprofen and is suitable encapsulation in a soft gelatin capsule.

Soft gelatin capsules offer a number of advantages over tablets and caplets as a pharmaceutical dosage form. They are easily digested and dissolve quickly in the stomach thus allowing quicker onset of the desired pharmaceutical effect. Soft gelatin capsules are also very useful for administering poorly soluble or poorly absorbed active pharmaceutical ingredients (API). Soft gelatin capsules are also useful for protecting APIs from light and oxygen thus improving their stability.

Soft gelatin capsules which contain a pain killer, such as ibuprofen, are known in the art and are commercially available.

U.S. Pat. No. 5,376,688 describes soft and hard gelatin capsules containing a fill formulation which comprises a diethylene glycol monoethyl ether and a polyglycerol oleate. U.S. Pat. No. 5,912,011 discloses a solvent system for encapsulation in soft and hard gelatin capsules. WO 88/02625 discloses a solvent system to enhance the solubility of APIs. WO 2005/123133 discloses a solvent system for APIs which comprises 15-50% by weight of polyethylene glycol.

However, while the soft gelatin capsules that are currently available exhibit a release rate and onset of pharmaceutical effect that is acceptable improvements in speed of action are continually sought after. The present invention provides a soft-gelatin capsule that works significantly quicker than current capsules.

According to an aspect of the present invention there is provided an composition for encapsulation in a soft gelatin shell which comprises ibuprofen, one or more polyvinyl pyrrolidones and one or more polyethylene glycols wherein the weight ratio of the ibuprofen to the one or more polyvinylpyrrolidones is from 2:1-15:1, the weight ratio of the ibuprofen to the one or more polyethylene glycols is from 1:1-5:1 and the weight ratio of the one or more polyethylene glycols to the one or more polyvinylpyrrolidones is 2:1-10:1.

Preferably the weight ratio of the ibuprofen to the one or more polyvinylpyrrolidones is 3:1-12:1. More preferably the weight ratio of the ibuprofen to the one or more polyvinylpyrrolidones is 4:1-8:1. Most preferably weight ratio of the ibuprofen to the one or more polyvinylpyrrolidones is 5:1-7:1.

Preferably the weight ratio of ibuprofen to the one or more polyethylene glycols is 1.2:1-3:1. More preferably the weight ratio of ibuprofen to the one or more polyethylene glycols is 1.8:1-2:1.

Preferably the weight ratio of the one or more polyethylene glycols to the one or more polyvinylpyrrolidones is 1.5:1-8.5:1. More preferably the weight ratio of the one or more polyethylene glycols to the one or more polyvinylpyrrolidones is 2:1-5:1. More preferably the weight ratio of the one or more polyethylene glycols to the one or more polyvinylpyrrolidones is 3:1-4:1.

Preferably the weight ratio of the ibuprofen to one or more polyvinylpyrrolidones is 5:1-7:1, the weight ratio of the ibuprofen to the one or more polyethylene glycols is 1.8:1-2:1 and the weight ratio of the one or more polyethylene glycols to the one or more polyvinylpyrrolidones is 3:1-4:1.

The composition can comprise ibuprofen, one or more polyethylene glycols and one or more polyvinylpyrrolidones wherein the weight ratio of the ibuprofen to the one or more polyethylene glycols to one or more polyvinylpyrrolidones is from about 15:10:1 to about 2:1:1.

Preferably the weight ratio of the ibuprofen: the one or more polyethylene glycols:one or more polyvinylpyrrolidones is from about 7:4:1 to about 6:3:1.

The composition can further include one or more polyoxysorbitan esters which can be selected from the group consisting of polysorbate 20, polysorbate 21, polysorbate 40, polysorbate 60, polysorbate 61, polysorbate 65, polysorbate 80, polysorbate 81, polysorbate 85 or polysorbate 120. A preferred polyoxysorbitan ester is polysorbate 80.

The composition can further comprise a base in an amount from about 1% w/w to about 15% w/w. Preferably, the base can be present at a level of from 5% w/w to 10% w/w. More preferably, the base can be present at a level of from about 6% w/w to about 9% w/w.

The base can be selected from alkali metal hydroxides (i.e. the metals of Group I of the Periodic Table) particularly sodium and potassium, and alkali earth metal hydroxides (i.e. the metals of Group II of the Periodic Table) particularly calcium and magnesium. Preferred hydroxides are sodium hydroxide and potassium hydroxide. A more preferred hydroxide is potassium hydroxide.

The base can also be selected from carbonate and bicarbonate salts of the alkali and alkali earth metals, i.e. the metals of Group I or Group II of the periodic table.

The base can be selected from amines and amino acids such as ammonia, triethylamine, lysine or arginine.

Typically, the one or more polyethylene glycols is a liquid at room temperature. The one or more polyethylene glycols can have a number average molecular weight (Mn) of up to 1000. The one or more polyethylene glycols can have a number average molecular weight (Mn) of from about 400 to about 800. Preferred one or more polyethylene glycols can have a number average molecular weight (Mn) of 200, 300, 400, 600, 800. More preferred one or more polyethylene glycols can have a number average molecular weight (Mn) of 400 or 600.

The the one or more polyvinylpyrrolidones can have a number average molecular weight (Mn) of up to 10,000. The one or more polyvinylpyrrolidones can have a number average molecular weight (Mn) of from about 2,000 to about 5,000. Preferably one or more polyvinylpyrrolidones can have a number average molecular weight (Mn) of 2,500-3,000. A preferred grade of polyvinylpyrrolidone has a K value of 10-15. A more preferred grade of polyvinylpyrrolidone has a K value of 12.

The composition can further comprise water in an amount from about 2% w/w to about 10% w/w. The water can be present at a level of from 3% w/w to 6% w/w.

Typically, the unit dose weight of the formulation is about 400-500 mg. More typically, the unit dose weight of the formulation is about 440-480 mg. Even more typically, the unit dose weight of the formulation is about 450-470 mg. Most typically, the unit dose weight of the formulation is about 460 mg.

Typically, the unit dose weight of the formulation is about 800-1000 mg. More typically, the unit dose weight of the formulation is about 850-950 mg. Even more typically, the unit dose weight of the formulation is about 900-940 mg. Most typically, the unit dose weight of the formulation is about 920-925 mg.

Typically, the unit dose has a fill volume of from 0.4 ml to 1 ml.

The composition can comprise an ibuprofen, one or more polyvinylpyrrolidones and one or more polyethylene glycols wherein ibuprofen is present at an amount of 40-50% w/w, the one or more polyvinylpyrrolidones is present at an amount of 0.1-20% w/w, and the one or more polyethylene glycols is present at an amount of 15-40% w/w and the weight ratio of the ibuprofen to the one or more polyvinylpyrrolidones is 5:1-7:1, the weight ratio of ibuprofen to the one or more polyethylene glycols is 1.2:1-2:1 and the weight ratio of the one or more polyethylene glycols to the one or more polyvinylpyrrolidones is 3:1-4.5:1.

The composition can comprise an ibuprofen, one or more polyvinylpyrrolidones, one or more polyethylene glycols and a base wherein ibuprofen is present at an amount of 40-50% w/w, the one or more polyvinylpyrrolidones is present at an amount of 0.1-20% w/w, and the one or more polyethylene glycols is present as at an amount of 15-40% w/w and the weight ratio of the ibuprofen to the one or more polyvinylpyrrolidones is 5:1-7:1, the weight ratio of ibuprofen to the one or more polyethylene glycols is 1.8:1-2:1 and the weight ratio of the one or more polyethylene glycols to the one or more polyvinylpyrrolidones is 3:1-4:1 and wherein the base is present at a level of from about 6% w/w to about 9% w/w.

The composition can comprise a poloxysorbitan selected from polysorbate 20, polysorbate 21, polysorbate 40, polysorbate 60, polysorbate 61, polysorbate 65, polysorbate 80, polysorbate 81, polysorbate 85 or polysorbate 120, a polyethylene glycol having a number average molecular weight (Mn) of from about 400 to about 800 and a polyvinylpyyrolidone having a number average molecular weight (Mn) of 2,000-5,000 or a K value of 10-15. Preferably the composition can comprise polysorbate 80, a polyethylene glycol can have a number average molecular weight (Mn) of 400 or 600, a polyvinyl pyrrolidone having a number average molecular weight (Mn) of 2,500-3,00 or a combination thereof.

According to an aspect of the present invention there is provided a composition comprising:
(a) 35-50% w/w ibuprofen;
(b) 0.1-20% w/w One or more polyvinylpyrrolidones; and
(c) 15-40% w/w One or more polyethylene glycols;
   wherein the volume of the unit dose of the composition is from about 0.4 ml to about 1 ml.

The composition can comprise:
(a) 40-45% w/w ibuprofen;
(b) 5-10% w/w One or more polyvinylpyrrolidones; and
(c) 15-30% w/w One or more polyethylene glycols;
   wherein the volume of the unit dose of the composition is from about 0.4 ml to about 1 ml.

The composition can comprise:
(a) 35-50% w/w ibuprofen;
(b) 2-20% w/w One or more polyvinylpyrrolidones;
(c) 1-15% w/w A hydroxide; and
(d) 15-40% w/w One or more polyethylene glycols.

The composition can comprise:
(a) 35-50% w/w ibuprofen;
(b) 2-20% w/w One or more polyvinylpyrrolidones;
(c) 1-15% w/w A hydroxide; and
(d) 15-40% w/w One or more polyethylene glycols
   wherein the volume of the unit dose of the composition is from about 0.4 ml to 1 ml.

The composition can consist essentially of:
(a) 35-50% w/w ibuprofen;
(b) 1-15% w/w One or more polyvinylpyrrolidones;
(c) 15-40% w/w One or more polyethylene glycols;
(d) 2-15% w/w A hydroxide; and
(e) 1-10% w/w Water.

The composition can consist essentially of:
(a) 35-50% w/w ibuprofen;
(b) 1-15% w/w One or more polyvinylpyrrolidones;
(c) 15-40% w/w One or more polyethylene glycols;
(d) 2-15% w/w A hydroxide; and
(e) 1-10% w/w Water
   wherein the volume of the unit dose of the composition is from about 0.4 ml to about 1 ml.

The composition can consist essentially of:
(a) 35-45% w/w ibuprofen;
(b) 5-15% w/w one or more polyvinylpyrrolidones;
(c) 30-35% w/w one or more polyethylene glycols;
(d) 4-10% w/w a hydroxide; and
(e) 3-8% w/w Water.

The composition can consist essentially of:
(a) 40-45% w/w ibuprofen;
(b) 5-10% w/w one or more polyvinylpyrrolidones;
(c) 30-35% w/w one or more polyethylene glycols;
(d) 5-10% w/w a hydroxide; and
(e) 3-8% w/w Water.

The composition can consist essentially of:
(a) 35-45% w/w ibuprofen;
(b) 3-10% w/w one or more polyvinylpyrrolidones;
(c) 15-35% w/w one or more polyethylene glycols;
(d) 1-20% w/w one or more polyoxysorbitan esters;
(e) 2-10% w/w A hydroxide; and
(f) 1-10% Water.

The composition can consist essentially of:
(a) 40-45% w/w ibuprofen;
(b) 5-10% w/w one or more polyvinylpyrrolidones;
(c) 15-25% w/w one or more polyethylene glycols;
(d) 15-20% w/w one or more polyoxysorbitan esters;
(e) 5-10% w/w a hydroxide; and
(f) 3-8% w/w water.

The composition can consist essentially of:
(a) 35-45% w/w ibuprofen;
(b) 3-10% w/w one or more polyvinylpyrrolidones;
(c) 15-35% w/w one or more polyethylene glycols;
(d) 1-20% w/w one or more polyoxysorbitan esters;
(e) 2-10% w/w a hydroxide; and
(f) 1-10% water
   wherein the volume of the unit dose of the composition is from about 0.4 ml to about 1 ml.

The composition can consist essentially of:
(a) 40-45% w/w ibuprofen;
(b) 5-10% w/w one or more polyvinylpyrrolidones;
(c) 15-25% w/w one or more polyethylene glycols;
(d) 15-20% w/w one or more polyoxysorbitan esters;
(e) 5-10% w/w a hydroxide; and
(f) 3-8% w/w water
   wherein the volume of the unit dose of the composition is from about 0.4 ml to about 1 ml.

The composition can comprise a poloxysorbitan selected from polysorbate 20, polysorbate 21, polysorbate 40, polysorbate 60, polysorbate 61, polysorbate 65, polysorbate 80, polysorbate 81, polysorbate 85 or polysorbate 120, a polyethylene glycol having a number average molecular weight (Mn) of from about 400 to about 800 and a polyvinylpyyrolidone having a number average molecular weight (Mn) of 2,000-5,000 or a K value of 10-15. Preferably the composition can comprise polysorbate 80, a polyethylene glycol can have a number average molecular weight (Mn) of 400 or 600, a polyvinyl pyrrolidone having a number average molecular weight (Mn) of 2,500-3,00 or a combination thereof.

According to an aspect of the present invention there is provided a soft gelatin capsule which contains a composition as described in any of the previous aspects.

According to an aspect of the present invention there is provided a soft gelatin capsule comprising a composition of ibuprofen as described in any of the previous aspects wherein the composition allows for at least 5% of the ibuprofen dose to be solubilised within 5 minutes of immersion of the soft gelatine capsule in simulated gastric fluid at a pH of 1.2.

Preferably the composition allows for at least 10% of the ibuprofen dose to be solubilised within 10 minutes of immersion of the soft gelatine capsule in simulated gastric fluid at a pH of 1.2. More preferably the composition allows for at least 10% of the ibuprofen dose to be solubilised within 5 minutes of immersion of the soft gelatine capsule in simulated gastric fluid at a pH of 1.2.

Preferably the composition allows for at least 15% of the ibuprofen dose to be solubilised within 10 minutes of immersion of the soft gelatine capsule in simulated gastric fluid at a pH of 1.2.

Preferably the composition allows for at least 20% of the ibuprofen dose to be solubilised within 10 minutes of immersion of the soft gelatine capsile in simulated gastric fluid at a pH of 1.2.

According to an aspect of the present invention there is provided a soft gelatin capsule comprising a composition of ibuprofen wherein the composition allows for at least 5% of the ibuprofen dose to be solubilised within 5 minutes of immersion of the soft gelatine capsule in simulated gastric fluid at a pH of 1.2.

Preferably the composition allows for at least 10% of the ibuprofen dose to be solubilised within 10 minutes of immersion of the soft gelatine capsule in simulated gastric fluid at a pH of 1.2. More preferably the composition allows for at least 10% of the ibuprofen dose to be solubilised within 5 minutes of immersion of the soft gelatine capsule in simulated gastric fluid at a pH of 1.2.

Preferably the composition allows for at least 15% of the ibuprofen dose to be solubilised within 10 minutes of immersion of the soft gelatine capsule in simulated gastric fluid at a pH of 1.2.

Preferably the composition allows for at least 20% of the ibuprofen dose to be solubilised within 10 minutes of immersion of the soft gelatine capsile in simulated gastric fluid at a pH of 1.2.

According to an aspect of the present invention there is provided a composition for a soft-gel capsule which comprises ibuprofen and a solvent system wherein the solvent system comprises:
a) from about 35 to about 65% w/w one or more polyethylene glycols;
b) from about 5 to about 30% w/w of one or more polyvinylpyrrolidones;
c) from about 5 to about 20% w/w of potassium hydroxide;
d) optionally 5-40% of one or more polyoxysorbitan esters; and
e) water.

Typically, the one or more polyoxysorbitan esters and the one or more polyethylene glycols are present at a combined level of 55-75% w/w.

The solvent system can consist essentially of:
a) from about 55 to about 60% w/w one or more polyethylene glycols;
b) from about 10 to about 30% w/w of one or more polyvinylpyrrolidones;
c) from about 5 to about 20% w/w of potassium hydroxide; and
d) water.

The solvent system can consist essentially of:
a) from about 55 to about 60% w/w one or more polyethylene glycols;
b) from about 10 to about 20% w/w of one or more polyvinylpyrrolidones;
c) from about 12 to about 20% w/w of potassium hydroxide; and
d) water.

The solvent system can consist essentially of:
a) from about 35 to about 60% w/w one or more polyethylene glycols;
b) from about 5 to about 15% w/w of one or more polyvinylpyrrolidones;
c) from about 5 to about 15% w/w of potassium hydroxide;
d) from about 5 to about 35% w/w of one or more polyoxysorbitan esters
e) water.

The solvent system can consist essentially of:
a) from about 35 to about 40% w/w one or more polyethylene glycols;
b) from about 10 to about 15% w/w of one or more polyvinylpyrrolidones;
c) from about 10 to about 15% w/w of potassium hydroxide;
d) from about 30 to about 35% w/w of one or more polyoxysorbitan esters
e) water.

Typically, the ibuprofen-containing composition has a weight of 440-960 mg. More typically, the composition has a weight of 450-940 mg. Most typically, the composition has a weight of 460-930 mg.

Typically, the composition has a release rate for ibuprofen of at least 5% in 5 mins. More typically, the composition has a release rate of at least 10% in 5 mins. Most typically, the composition has a release rate of at least 20% in 5 mins.

The composition can comprise a poloxysorbitan selected from polysorbate 20, polysorbate 21, polysorbate 40, polysorbate 60, polysorbate 61, polysorbate 65, polysorbate 80, polysorbate 81, polysorbate 85 or polysorbate 120, a polyethylene glycol having a number average molecular weight (Mn) of from about 400 to about 800 and a polyvinylpyyrolidone having a number average molecular weight (Mn) of 2,000-5,000 or a K value of 10-15. Preferably the composition can comprise polysorbate 80, a polyethylene glycol can have a number average molecular weight (Mn) of 400 or 600, a polyvinyl pyrrolidone having a number average molecular weight (Mn) of 2,500-3,00 or a combination thereof.

According to an aspect of the present invention there is provided a soft-gel capsule which contains a composition which can comprise ibuprofen and a solvent system wherein the solvent system can consist essentially of:
a) from about 55 to about 60% w/w of one or more polyethylene glycols;
b) from about 10 to about 20% w/w of one or more polyvinylpyrrolidones;
c) from about 12 to about 20% w/w of potassium hydroxide; and
d) water.
   wherein the composition has a weight of 440-480 mg and wherein the composition has a release rate for ibuprofen of at least 5% in 5 mins.

The composition can comprise a polyethylene glycol having a number average molecular weight (Mn) of from about 400 to about 800 and a polyvinylpyyrolidone having a number average molecular weight (Mn) of 2,000-5,000 or a K value of 10-15. Preferably the composition can comprise a polyethylene glycol can have a number average molecular weight (Mn) of 400 or 600 and a polyvinyl pyrrolidone having a number average molecular weight (Mn) of 2,500-3,00 or a combination thereof.

According to an aspect of the present invention there is provided a soft-gel capsule which contains a composition which can comprise ibuprofen and a solvent system wherein the solvent system can consist essentially of:
a) from about 35 to about 40% w/w of one or more polyethylene glycols;
b) from about 10 to about 15% w/w of one or more polyvinylpyrrolidones;
c) from about 10 to about 15% w/w of potassium hydroxide;
d) from about 30 to about 35% w/w of one or more polyoxysorbitan esters
e) water
   wherein the composition has a weight of 440-480 mg and wherein the composition has a release rate for ibuprofen of at least 5% in 5 mins.

The composition can comprise a poloxysorbitan selected from polysorbate 20, polysorbate 21, polysorbate 40, polysorbate 60, polysorbate 61, polysorbate 65, polysorbate 80, polysorbate 81, polysorbate 85 or polysorbate 120, a polyethylene glycol having a number average molecular weight (Mn) of from about 400 to about 800 and a polyvinylpyyrolidone having a number average molecular weight (Mn) of 2,000-5,000 or a K value of 10-15. Preferably the composition can comprise polysorbate 80, a polyethylene glycol having a number average molecular weight (Mn) of 400 or 600, a polyvinyl pyrrolidone having a number average molecular weight (Mn) of 2,500-3,00 or a combination thereof.

As used herein the reference to the solubilisation of ibuprofen refers to the release and solubilisation of an amount of the ibuprofen dose in simulated gastric fluid. For example, when the composition allows for the release and solubilisation of 5% of the ibuprofen dose within 5 minutes this means that within 5 minutes of immersion in simulated gastric fluid 5% of the ibuprofen dose has been is in solution. For example, if the ibuprofen dose is 100 mg then 5 mg of the ibuprofen will have been both released from the capsule and solubilised. An additional amount may have been released but will not have been solubilised within the 5 minute period following immersion of the gelatin capsule.

As used herein the reference to weight ratios refers to the percentage weight of the components in the composition. For example, a composition having a 40% by weight of a first component and 40% by weight of a second component has a weight ratio of 1:1.

As used herein the reference to the unit dose weight of the formulation refers to weight of the composition that would be filled into the gelatine shell. The terms ‘formulation’ and ‘composition’ are used interchangeably and have the same meaning.

For the avoidance of doubt, in accordance with the present invention it is the compositions which are for encapsulation within a gelatin shell that comprise the ibuprofen, base, polyoxysorbitan ester or polyethylene glycol and not in the gelatin shell itself.

For the avoidance of doubt, the compositions described in the present specification are in the form of a liquid.

Embodiments of the invention will now be described by way of example only with reference to the accompanying FIGURE in which:

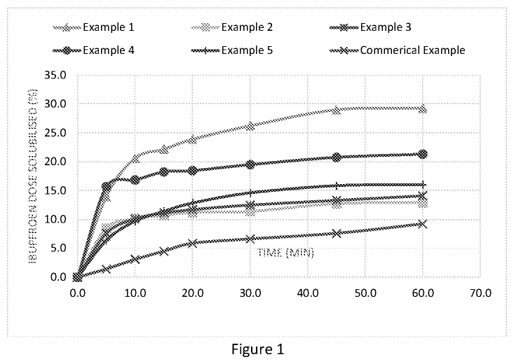

FIG. 1 illustrates release profiles for ibuprofen from compositions of the present invention and current commercially available soft gelatin capsules.

The following Table illustrates examples of the composition of the present invention.

| | Example 1 | Example 2 | Example 3 | Example 4 | Example 5 |
|---|---|---|---|---|---|
| Component | (% w/w) | (% w/w) | (% w/w) | (% w/w) | (% w/w) |
| Ibuprofen | 43.38 | 43.38 | 43.38 | 43.38 | 43.38 |
| Polysorbate | — | — | 17.54 | 19.30 | 5.09 |
| 80 | | | | | |
| PEG 400 | 33.75 | 33.40 | 21.77 | 22.93 | 34.10 |
| KOH Pellets | 4.72 | 8.93 | 6.48 | 4.72 | 7.98 |
| Water | 3.15 | 5.95 | 4.34 | 3.15 | 5.32 |
| Kollidon | 15.00 | 8.34 | 6.49 | 6.52 | 4.13 |
| Total | 100.00 | 100.00 | 100.00 | 100.00 | 100.00 |

The formulation can be made using standard techniques known to the person of ordinary skill in the art. Kollidon and polyethylene glycol and/or polysorbate are mixed together while heating to a temperature of 55° C. A portion of ibuprofen is added followed by a portion of aqueous potassium hydroxide solution while maintaining the heating. Once a homogenous solution has been achieved, the remaining ibuprofen and aqueous potassium hydroxide solution are added with continued heating. The resulting mixture is then stirred until a clear solution is obtained. The resulting solution can be stored until required for encapsulation in a gelatin capsule.

Similarly, the formulation can be encapsulated using standard soft gelatin encapsulation techniques well-known to the person skilled in the art. For example, the composition of the present invention can be deposited between two opposing ribbons of a gel composition. The composition of the ribbons may include gelatin and plasticisers such as glycerine and sorbitol and mixtures thereof. For example, a suitable gel composition can comprise about 35-50% by weight of gelatin, 20-40% by weight of a plasticiser and 25-50% by weight of water. The opposing ribbons are then run between two die rollers having die pockets thereon the surface of which corresponds to the configuration of the desired soft capsule. The composition is sealed within the fused casing.

The soft gelatin capsules were filled with compositions containing the standard dose of 200 mg of ibuprofen, i.e. in the examples above the amount of ibuprofen is 200 mg.

The release rate of the compositions of the present invention were determined by immersing the compositions in a soft gelatin capsule in simulated gastic fluid. The compositions were immersed into a beaker/container containing 900 ml of the simulatied gastric fluid at a temperature of 37° C. The pH of the simulated gastric fluid was 1.2.

The results are shown in the following Table and in FIG. 1.

| | Exam- | Exam- | Exam- | Exam- | Exam- | Commercial |
|---|---|---|---|---|---|---|
| Time | ple 1 | ple 2 | ple 3 | ple 4 | ple 5 | Example |
| 0.0 | 0.0 | 0.0 | 0 | 0 | 0 | 0 |
| 5.0 | 14.0 | 8.5 | 7.6 | 15.7 | 6.4 | 1.43 |
| 10.0 | 20.6 | 10.2 | 10.1 | 16.8 | 9.7 | 3.1 |
| 15.0 | 22.2 | 10.7 | 11.1 | 18.2 | 11.4 | 4.5 |
| 20.0 | 23.9 | 11.2 | 11.7 | 18.5 | 12.85 | 5.87 |
| 30.0 | 26.2 | 11.4 | 12.5 | 19.5 | 14.6 | 6.63 |
| 45.0 | 29.0 | 12.8 | 13.3 | 20.8 | 15.85 | 7.6 |
| 60.0 | 29.3 | 12.9 | 14.1 | 21.3 | 16 | 9.23 |

As can be seen from the Table and FIG. 1, the composition/pharmaceutical product of the present invention releases the ibuprofen unexpectedly and significantly quicker than a current commercial product tested under the same conditions.

An advantage of the present invention is that there is provided a stable ibuprofen-containing formulation which is suitable for encapsulation in a soft gelatin capsule and which releases the ibuprofen in a more solubilisable form than standard ibuprofen-containing soft gelatin capsules. A further advantage is provided with the ability to use a lower amount of base within the formulation whilst improving the solubilisation of the Ibuprofen in simulated gastric fluid.

Further modifications can be made without departing from the scope of the invention described herein.

1-81. (canceled)

82. A composition for encapsulation in a soft gelatin shell, the composition comprising:
ibuprofen;
one or more polyvinyl pyrrolidones; and
one or more polyethylene glycols,
wherein a weight ratio of the ibuprofen to the one or more polyvinylpyrrolidones is from 2:1-15:1, a weight ratio of the ibuprofen to the one or more polyethylene glycols is from 1:1-5:1, and a weight ratio of the one or more polyethylene glycols to the one or more polyvinylpyrrolidones is 2:1-10:1.

83. The composition as claimed in claim 82, wherein the weight ratio of the ibuprofen to the one or more polyvinylpyrrolidones is 3:1-12:1.

84. The composition as claimed in claim 82, wherein the weight ratio of the ibuprofen to the one or more polyvinylpyrrolidones is 5:1-7:1.

85. The composition as claimed in claim 82, wherein the weight ratio of ibuprofen to the one or more polyethylene glycols is 1.2:1-3:1.

86. The composition as claimed in in claim 82, wherein the weight ratio of the one or more polyethylene glycols to the one or more polyvinylpyrrolidones is 1.5:1-8.5:1.

87. The composition as claimed in claim 86, wherein the weight ratio of the one or more polyethylene glycols to the one or more polyvinylpyrrolidones is 3:1-4:1.

88. The composition as claimed in claim 82, wherein the weight ratio of the ibuprofen to one or more polyvinylpyrrolidones is 5:1-7:1, the weight ratio of the ibuprofen to the one or more polyethylene glycols is 1.8:1-2:1, and the weight ratio of the one or more polyethylene glycols to the one or more polyvinylpyrrolidones is 3:1-4:1.

89. The composition as claimed in claim 82, wherein the weight ratio of the ibuprofen to the one or more polyethylene glycols to the one or more polyvinylpyrrolidones is from about 15:10:1 to about 2:1:1.

90. The composition as claimed in claim 89, wherein the weight ratio of the ibuprofen to the one or more polyethylene glycols to the one or more polyvinylpyrrolidones is from about 7:4:1 to about 6:3:1.

91. The composition as claimed in claim 82, wherein the composition further comprises a base in an amount of from 5% w/w to 10% w/w.

92. The composition as claimed in claim 82, wherein the composition further comprises water in an amount from about 2% w/w to about 10% w/w.

93. The composition as claimed in claim 92, wherein the water is present in an amount of from 3% w/w to 6% w/w.

94. The composition as claimed in claim 82, wherein the composition comprises:
40-50% w/w of the ibuprofen;
0.1-20% w/w of the one or more polyvinylpyrrolidones; and
15-40% w/w of the one or more polyethylene glycols,
wherein the weight ratio of the ibuprofen to the one or more polyvinylpyrrolidones is 5:1-7:1, the weight ratio of the ibuprofen to the one or more polyethylene glycols is 1.2:1-2:1, and the weight ratio of the one or more polyethylene glycols to the one or more polyvinylpyrrolidones is 3:1-4.5:1.

95. The composition as claimed in claim 94, wherein the composition comprises:
40-50% w/w of the ibuprofen;
0.1-20% w/w of the one or more polyvinylpyrrolidones;
15-40% w/w of the one or more polyethylene glycols; and
6% w/w to about 9% w/w of a base,
wherein the weight ratio of the ibuprofen to the one or more polyvinylpyrrolidones is 5:1-7:1, the weight ratio of ibuprofen to the one or more polyethylene glycols is 1.8:1-2:1 and the weight ratio of the one or more polyethylene glycols to the one or more polyvinylpyrrolidones is 3:1-4:1.

96. A composition consisting essentially of:
35-45% w/w of ibuprofen;
3-10% w/w of one or more polyvinylpyrrolidones;
15-35% w/w of one or more polyethylene glycols;
1-20% w/w of one or more polyoxysorbitan esters;
2-10% w/w of a hydroxide; and
1-10% w/w water.

97. The composition as claimed in claim 96, wherein the composition consists essentially of:
40-45% w/w of ibuprofen;
5-10% w/w of one or more polyvinylpyrrolidones;
15-25% w/w of one or more polyethylene glycols;
15-20% w/w of one or more polyoxysorbitan esters;
5-10% w/w of the hydroxide; and
3-8% w/w of water.

98. A composition for a soft-gel capsule, the composition comprising:
ibuprofen; and
a solvent system, the solvent system comprising:
from about 35 to about 65% w/w one or more polyethylene glycols;
from about 5 to about 30% w/w of one or more polyvinylpyrrolidones;
from about 5 to about 20% w/w of potassium hydroxide;
optionally 5-40% of one or more polyoxysorbitan esters; and
water.

99. The composition as claimed in claim 98, wherein the one or more polyoxysorbitan esters and the one or more polyethylene glycols are present at a combined level of 55-75% w/w.

100. The composition as claimed in claim 99, wherein the solvent system consists essentially of:
from about 35 to about 60% w/w one or more polyethylene glycols;
from about 5 to about 15% w/w of one or more polyvinylpyrrolidones;
from about 5 to about 15% w/w of potassium hydroxide;
from about 5 to about 35% w/w of one or more polyoxysorbitan esters; and
water.

101. A soft gelatin capsule comprising the composition as claimed in claim 82.

102. The composition as claimed in claim 82, wherein the poloxysorbitan ester is selected from polysorbate 20, polysorbate 21, polysorbate 40, polysorbate 60, polysorbate 61, polysorbate 65, polysorbate 80, polysorbate 81, polysorbate 85, and polysorbate 120,
wherein the polyethylene glycol has a number average molecular weight (Mn) of from about 400 to about 800, and
wherein the polyvinylpyyrolidone has a number average molecular weight (Mn) of 2,000-5,000 or a K value of 10-15.
